(19) United States
(12) Patent Application Publication
(10) Pub. No.: US 2016/0325282 A1
(43) Pub. Date: Nov. 10, 2016

(54) LIQUID SPECIMEN SENSOR, LIQUID SPECIMEN SENSOR UNIT, AND LIQUID SPECIMEN TESTING METHOD

(71) Applicants: KYOCERA CORPORATION, Kyoto (JP); OSAKA UNIVERSITY, Osaka (JP)

(72) Inventors: Hideharu KURIOKA, Kyoto (JP); Hiroyasu TANAKA, Kyoto (JP); Masato SAITO, Osaka (JP); Eiichi TAMIYA, Osaka (JP)

(21) Appl. No.: 15/108,154

(22) PCT Filed: Dec. 26, 2014
(86) PCT No.: PCT/JP2014/084658

§ 371 Date: Jun. 24, 2016

(30) Foreign Application Priority Data
Dec. 26, 2013 (JP) 2013-270277

(51) Int. Cl. B01L 3/00; G01N 29/22; G01N 33/49; G01N 29/02

(52) U.S. Cl. CPC B01L 3/502753; G01N 29/022; G01N 29/222; G01N 33/49; G01N 2291/02466; G01N 2291/0423; B01L 2300/0681; B01L 2300/0816; B01L 2400/0406

(57) ABSTRACT

A liquid specimen sensor has a filter filtering a liquid specimen, a first channel which is located downstream relative to the filter and is formed from a capillary, a second channel which is located downstream relative to the filter and is formed from a capillary, and a sensor element which is located in the second channel and outputs a signal in accordance with an ingredient of the liquid specimen.

### TECHNICAL FIELD

The present invention relates to a sensor capable of measuring a property of a liquid or an ingredient contained in the liquid, a sensor unit including the sensor, and a testing method using the sensor. Note that, the liquid has only to be one having fluidity, and may have a high viscosity.

### BACKGROUND ART

Known in the art is the technique of separating blood to blood plasma and blood cells by a membrane filter in order to test the blood (for example Patent Literature 1).

A test kit in Patent Literature 1 has a membrane filter, first and second containers which are connected through channels to the membrane filter, and a cock for controlling the flow from the membrane filter to the first and second containers. In the first and second containers, reagents which are different from each other are placed.

In the test kit in Patent Literature 1, first, the cock is set so that the liquid flows from the membrane filter to the first container, and the blood is supplied to the membrane filter. Due to this, the blood plasma is held in the first container, while the blood cells remain at the membrane filter. The blood plasma reacts with the reagent arranged in the first container.

Next, the cock is set so that the liquid flows from the membrane filter to the second container, and purified water is supplied to the membrane filter. Due to this, the blood cells remaining at the membrane filter are hemolyzed and are held in the second container. The hemolyzed blood cells react with the reagent which is arranged in the second container.

In the technique in Patent Literature 1, the first and second containers are arranged below the membrane filter, and the channels connecting the membrane filter with the first and second containers are constituted by tubes. The flow from the membrane filter to the first and second containers is realized by gravity.

In Patent Literature 1, by providing the membrane filter in the test kit, separation of the blood outside of the test kit is made unnecessary, therefore a simplified testing method is realized. In Patent Literature 1, however, the liquid specimen is made flow by gravity. Therefore, for example, when using the test kit, a constant direction must be set downward, so there are restrictions in handling. Further, in Patent Literature 1, the flow is controlled by the cock. Therefore, for example, the test kit is apt to become large in size in configuration. In this way, the test kit becomes complex or large in size in configuration or restrictions arise in the testing method.

Therefore, it is desired to provide a liquid specimen sensor, liquid specimen sensor unit, and testing method capable of effectively controlling the flow of the liquid specimen and capable of realizing simple tests.

### CITATION LIST

### Patent Literature

Patent Literature 1: Japanese Patent Publication No. 2012-88089A

### SUMMARY OF INVENTION

A liquid specimen sensor according to one aspect of the present invention has a filter filtering a liquid specimen, a first channel which is located downstream relative to the filter and is formed from a capillary, a second channel which is located downstream relative to the filter and is formed from a capillary, and a sensor element which is located in at least one of the first channel and the second channel and outputs a signal in accordance with an ingredient of the liquid specimen.

A liquid specimen sensor unit according to an aspect of the present invention has the above liquid specimen sensor and a reader to which the liquid specimen sensor can be attached and from which it can be detached.

A liquid specimen testing method according to an aspect of the present invention has, for a liquid specimen sensor having a filter filtering a liquid specimen, a first channel and second channel which are located downstream relative to the filter and are formed from capillaries, and a sensor element which is located in the second channel and outputs a signal in accordance with an ingredient of the liquid specimen, a first supplying step of supplying the liquid specimen to the filter in a state where a position in the second channel which is located more downstream than the sensor element and an external portion are not communicated; a first communication step of communicating a first portion in the flow direction in the first channel and the external portion; a second supplying step of supplying a liquid capable of reacting with the liquid specimen to the filter after the first supplying step and the first communication step; and a second communication step of communicating a position in the second channel which is located more downstream than the sensor element and an external portion after the first supplying step and the first communication step.

According to the above constitution or procedure, the flow of the liquid specimen can be effectively controlled, and a simplified test can be realized.

### BRIEF DESCRIPTION OF DRAWINGS

FIG. 1 A perspective view showing a sensor according to an embodiment of the present invention.

FIG. 2 A broken down perspective view of the sensor in FIG. 1.

FIG. 3A is a top view showing the sensor after detaching an upper layer member from the sensor in FIG. 1, FIG. 3B is a cross-sectional view of the sensor taken along a line IIIb-IIIb in FIG. 3A, and FIG. 3C is a cross-sectional view of the sensor taken along a line IIIc-IIIc in FIG. 3A.

FIG. 4 A plan view showing a sensor element in the sensor in FIG. 1.

FIG. 5A to FIG. 5E are diagrams for explaining the procedure of a liquid specimen testing method using the sensor in FIG. 1.

FIG. 6A to FIG. 6E are diagrams for explaining the continuation of FIG. 5E.

FIG. 7A and FIG. 7B are cross-sectional views showing examples in which capillary forces of the first channel and second channel are made different from each other.

FIG. 8A to FIG. 8D are diagrams showing various modifications of the shapes of the first channel and second channel.

FIG. 9A to FIG. 9C are diagrams showing various modifications of the shapes of the first channel and second channel.

FIG. 10A to FIG. 10C are diagrams for explaining a sensor unit including the sensor in FIG. 1.

FIG. 11 A diagram showing a modification of the first channel

### DESCRIPTION OF EMBODIMENTS

Below, an embodiment of a sensor according to the present invention will be explained in detail with reference to the drawings. Note that, in the drawings explained below, the constitutions which are the same or similar will be assigned the same notations. Note that, in the drawings, portions will be diagrammatically shown, and sizes etc. of the portions will sometimes differ from the actual ones.

Further, in the sensor, any direction may be defined as “upward” or “downward”. In the following description, however, for convenience, an orthogonal coordinate system xyz will be defined, and an “upper surface”, “lower surface”, and other terms will be used where the positive side of the z-direction is the upper part.

(Configuration of Sensor)

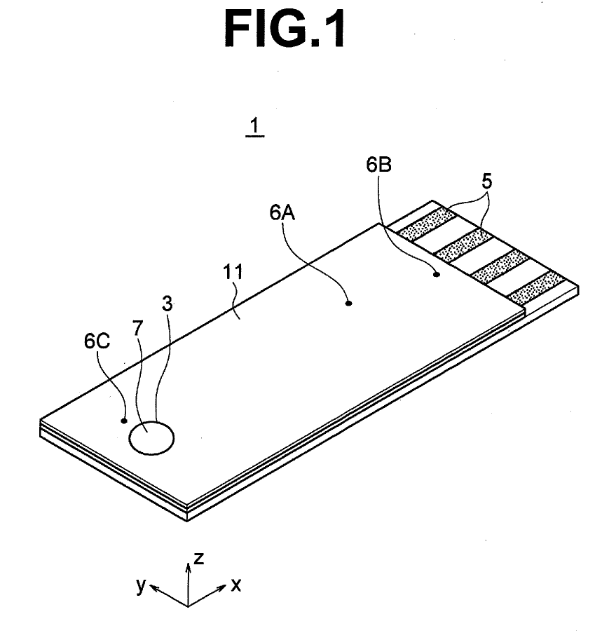

FIG. 1 is a perspective view showing a sensor 1 (liquid specimen sensor) according to an embodiment of the present invention.

The sensor 1 is for example formed in a roughly square plate shape as a whole. The thickness thereof is for example 0.5 mm to 3 mm, the length in the x-direction is for example 1 cm to 5 cm, and the length in the y-direction is for example 1 cm to 3 cm.

The sensor 1 is provided with an inflow port 3 for taking in a liquid specimen (for example blood or diluted blood) and a plurality of terminals which are provided for input and output of electrical signals.

The inflow port 3 is for example opened at an upper surface of the sensor 1 and at one end of the square. Note that, at the stage of distribution of the sensor 1 (before use of the sensor 1), for the purpose of suppression of entry of dust or unintended moisture from the inflow port 3 and so on, the inflow port 3 may be closed by a seal etc. as well. The shape of the inflow port 3 may be suitably set. However, it is for example a circle. The plurality of terminals 5 are for example exposed on the upper surface side of the sensor 1 at the other end of the square.

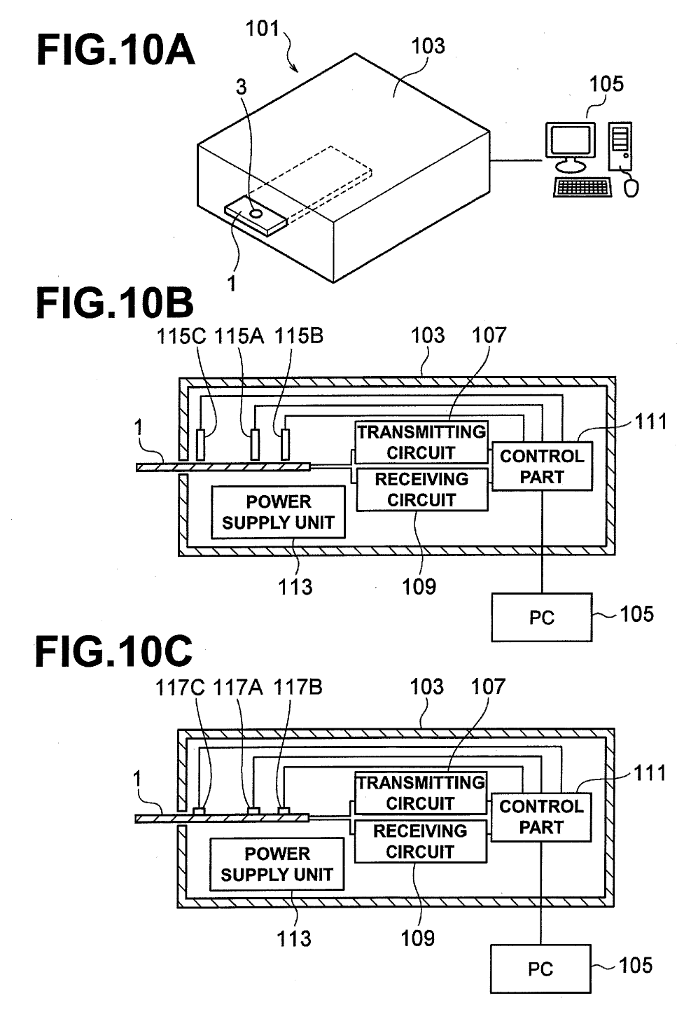

The sensor 1 is for example mounted on a reader 103 (see FIG. 10A) including an oscillation circuit and so on. It is mounted by for example inserting the end part on the terminal 5 side of the sensor 1 into a slot of the reader 103. Further, the sensor 1 changes the electrical signal which is input from the reader 103 to any among the plurality of terminals 5 in accordance with an ingredient of the liquid specimen taken from the inflow port 3 and outputs the result from any of the plurality of terminals 5 to the reader 103. The sensor 1 is for example a disposable sensor.

The upper surface of the sensor 1 is provided with a first mark 6A, second mark 6B, and third mark 6C (below, sometimes simply referred to as the “marks 6” and not discriminated). As will be explained later, in the upper surface of the sensor 1, holes are formed by the user. The marks 6 indicate the positions at which those holes must be opened.

The marks 6 may be given suitable forms so far as they can be confirmed by sight. For example, the marks 6 may be given a color which is different from that on the periphery so as to be able to be confirmed by sight or may be given irregularities so as to be able to be confirmed by sight. Further, the marks 6 may show positions at which holes must be opened by provision at the positions or may show positions at which holes must be opened even when they are deviated from the positions such as with arrows showing the positions. Note that, these marks 6 also need not be provided.

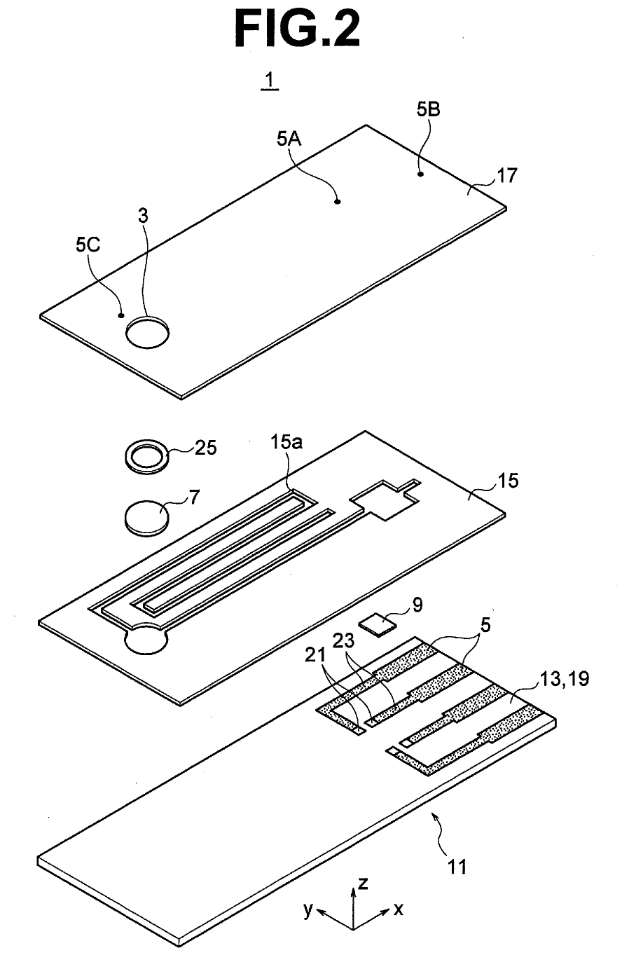

FIG. 2 is a broken down perspective view of the sensor 1.

The sensor 1 has a filter 7 for filtering the liquid specimen, a sensor element 9 which outputs a signal in accordance with an ingredient of the liquid specimen passed through the filter 7, and a package 11 (constituted by 13, 15, 17, etc.) which holds them and forms channels in which the liquid specimen flows.

The filter 7 is for example constituted by a so-called “membrane filter”. The membrane filter is for example a porous material (generally a porous membrane) which is mainly comprised of a fluororesin or cellulose acetate and has pore sizes contained in a relatively narrow range (the pore sizes are even). Accordingly, the filter 7 permits passage of an ingredient having a diameter not more than a predetermined diameter, but suppresses or prohibits the passage of an ingredient having a diameter larger than the predetermined diameter.

For example, the liquid specimen contains blood, and the pore size of the filter 7 is smaller than the diameter of the red blood cells of that blood. Further, the filter 7 permits the blood plasma to pass therethrough, but suppresses or prohibits the passage of the red blood cells. Note that, suppression or prohibition of passage is a concept including a case where the red blood cells pass through a portion on the front surface side of the filter 7 and remain inside the filter 7. Further, in a state where the red blood cells remain in the filter 7, when water (H₂O, for example, purified water) is supplied to the filter 7 and the red blood cells are hemolyzed, the filter 7 permits the passage of the hemolyzed ingredient of the red blood cells.

Note that, the shape of the red blood cells is roughly a disk shape in which a concave portion is formed. The diameter of the red blood cells referred to here relates to whether the cells can pass through the filter 7. Therefore, the diameter referred to here is for example the maximum diameter in the schematically disk shape. It is actually rare to set the pore size of the filter 7 close to the diameter of the red blood cells, therefore peculiar deformation of red blood cells need not be considered. Further, the diameter of the red blood cells used as the standard when setting the pore size may be determined for each animal (including humans) being subject. There is variation in the diameters of a plurality of blood cells. However, the diameter of the red blood cells which is used as the standard may be determined as for example the lower limit value of a general range of diameters of red blood cells of the animal (may be represented by humans as well) being subject. Note that, the fine sorting described above need not be considered, but the pore size may be made sufficiently small relative to the general size of red blood cells (for example less than 1 qua).

The hole shape of the filter 7 does not become a perfect circle when viewed in a penetration direction. However, the pore size referred to here is for example the maximum diameter from the viewpoint of securing the collection property. Note, in general, in the filter 7 (particularly a membrane filter), the hole shape is close to a circle, therefore the maximum diameter need not be an issue in many cases. Further, there is some variation in the diameter among a plurality of holes in one filter 7. However, for example, a mean value may be used as the pore size. From the viewpoint of securing the collection property, the lower limit value of the range in which most of the pore sizes in the filter 7 are contained may be used as well. The pore size may be found according to a collection test, porosimeter method, bubble point, or another known method.

The filter 7 is for example arranged at a position where it is exposed from the inflow port 3 to the outside of the package 11. For example, the filter 7 is arranged in the inflow port 3 and/or just beneath it. The shape and size of the filter 7 may be suitably set. For example, the filter 7 is given a flat plate shape in which the planar shape and size are roughly same as them of the inflow port 3 (disk shape in the present embodiment).

The sensor element 9 is for example formed in a roughly cuboid shape. The sensor element 9 is connected to the plurality of terminals 5. The sensor element 9 receives as input a signal from any of the plurality of terminals 5 and outputs a signal to any of the plurality of terminals 5.

The package 11 for example has a lower layer member 13, middle layer member 15, and upper layer member 17 (has a plurality of base substrates) which have layer shapes and are stacked in that order from the lower side. In the middle layer member 15, a groove 15a (slit) is formed which extends along its major surface. Due to this, between the lower layer member 13 and the upper layer member 17, a space for accommodating the filter 7 and sensor element 9 and a channel for guiding the liquid specimen are constituted.

The lower layer member 13 is for example given the same constitution as that of a printed circuit board. An insulating base 19 thereof is for example constituted by a resin or ceramic as the main body. The planar shape of the insulating base 19 is for example the same as the planar shape of the entire sensor 1. On the upper surface of the insulating base 19, the filter 9 and sensor element 9 are arranged. In the lower layer member 13, on the upper surface thereof, the already explained plurality of terminals 5, a plurality of pads 21 which are connected to the sensor element 9, and a plurality of lines 23 for connecting the plurality of terminals 5 and the plurality of pads 21 are provided. The arrangements and shapes of these may be suitably set.

The middle layer member 15 is for example constituted by an insulating material such as a resin or ceramic and is adhered to the lower layer member 13 and upper layer member 17 by an adhesive. Note that, the middle layer member 15 may be constituted by a double-sided tape using PET as the base material as well. The planar shape (schematic shape) of the middle layer member 15 is set to a square which is a bit shorter than the lower layer member 13 so that the plurality of terminals 5 are exposed. The shape of a groove 15a (channel etc.) will be explained later.

The upper layer member 17 is for example constituted by a hydrophilic film. The planar shape of the upper layer member 17 is set to a square which is a bit shorter than the lower layer member 13 in the same way as the middle layer member 15. Further, in the upper layer member 17, the already explained inflow port 3 and marks 6 are formed.

As the hydrophilic film, use can be made of a commercially available hydrophilized resin film. As the hydrophilization, for example, there can be mentioned a method of arranging (fixing) a coating agent. More specifically, for example, ashing by oxygen plasma may be carried out with respect to the base material (resin film), a silane coupling agent may be coated, and a polyethylene glycol as a coating agent may be coated. Also, for example, surface treatment may be carried out with respect to the base material by using a treatment agent having phosphorylcholine, and a coating agent comprised of phosphorylcholine may be fixed as well. The resin film is for example comprised of polyester-based or polyethylene-based resin.

The hydrophilicity (wettability with respect to the liquid specimen) can be measured by the degree of the contact angle with the liquid specimen (may be represented by water, same true for the following description) as generally known. That is, the higher the wettability, the smaller the contact angle. The contact angle of the hydrophilic film with the liquid specimen is less than 90°, preferably less than 60°, more preferably less than 10°.

The sensor 1, for example, does not have flexibility. For example, at least one (for example, the lower layer member 13) of the lower layer member 13, middle layer member 15, and upper layer member 17 does not have flexibility. However, the sensor 1 may have flexibility.

A double-sided tape 25 may be arranged between the filter 7 and the upper layer member 17. The planar shape of the double-sided tape 25 is a ring shape (the “ring shape” referred to here is not limited to a circle). This ring-shaped double-sided tape 25 has for example a constant width. The shapes of its inner circumferential edge and outer circumferential edge are similar to that of the inflow port 3 (filter 7).

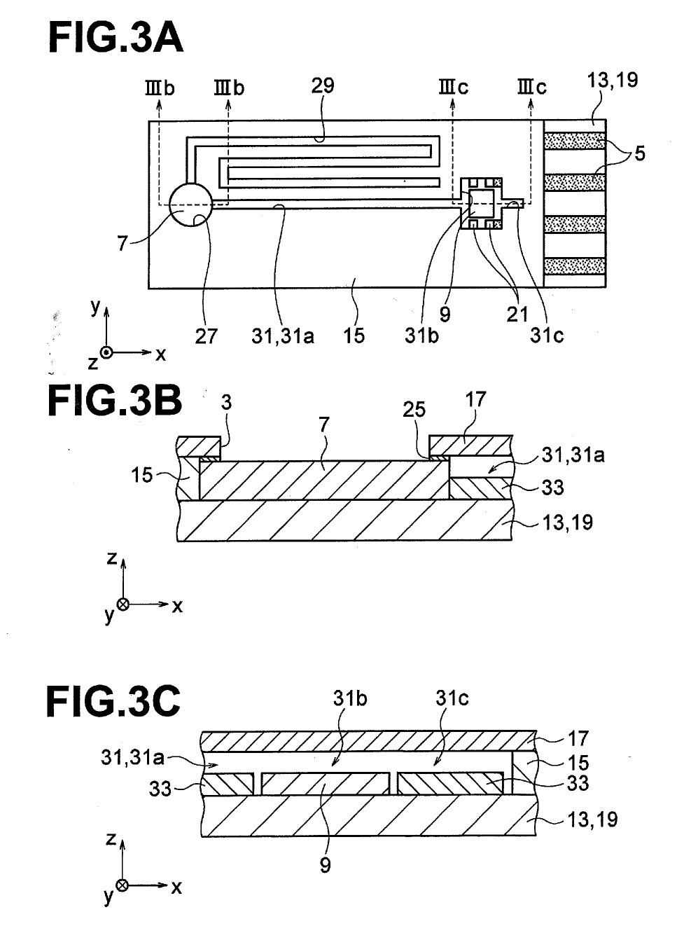

FIG. 3A is a top view of the sensor 1 shown by detaching the upper layer member 17 from the sensor 1.

As explained above, the groove 15a is formed in the middle layer member 15. Due to this, various spaces and channels are formed between the lower layer member 13 and the upper layer member 17. Specifically, a filter arrangement portion 27 for arranging the filter 7, and a first channel 29 and second channel 31 which extend from the filter arrangement portion 27 are formed.

The filter arrangement portion 27 is for example shaped so that the filter 7 is fit therein and is circular in the present embodiment.

The first channel 29 is for example a channel for retracting an ingredient (for example blood plasma) which is not a measurement object (test object) by the sensor element 9 in the liquid specimen supplied to the filter 7. In the present embodiment, the first channel 29 is directly connected to the filter 7. The shape and size of the first channel 29 may be suitably set. However, the shape and size are preferably set so as to secure a capacity large enough to hold an ingredient etc. which are not the measurement object. For example, in the present embodiment, the first channel 29 is given a roughly constant width (cross-sectional area) and extends so as to bend zigzag for securing the capacity. That is, the first channel 29 has a plurality of bent parts bending in the flow direction.

The second channel 31 is for example a channel for guiding the ingredient which is the measurement object by the sensor element 9 (for example, ingredient of hemolyzed blood cells) in the liquid specimen supplied to the filter 7 to the sensor element 9. In the present embodiment, the second channel 31 is directly connected to the filter 7. The second channel 31 for example has an inflow portion 31a extending from the filter arrangement portion 27, an element arrangement portion 31b which is connected to the inflow portion 31a and in which the sensor element 9 is arranged, and an outflow portion 31c which extends from the element arrangement portion 31b to the side opposite to the inflow portion 31a side.

The shapes and sizes of the portions in the second channel 31 may be suitably set. Note, the shapes and sizes are preferably set so that the required quantity of the liquid specimen becomes small. For example, in the present embodiment, the inflow portion 31a is given a width narrower than that of the element arrangement portion 31b (sensor element 9) and linearly extends thereby reducing the required quantity of the liquid specimen. Note that, the inflow portion 31a, element arrangement portion 31b, and outflow portion 31c may be given widths the same as each other as well.

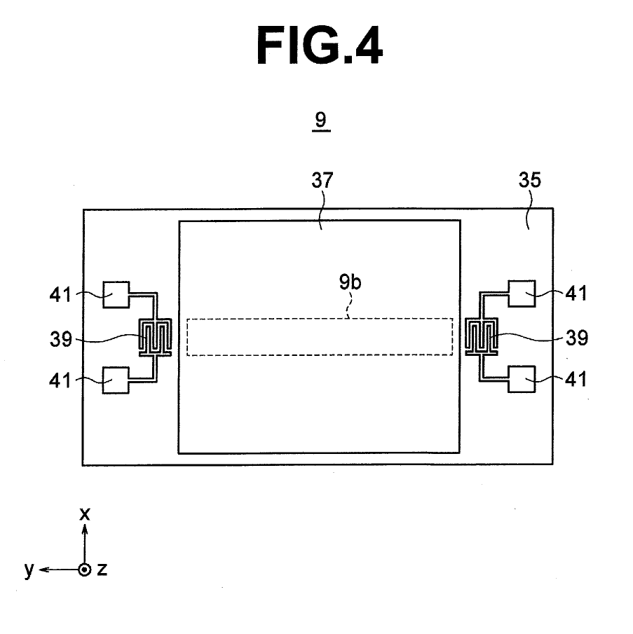

In the element arrangement portion 31b, the already explained plurality of pads 21 are exposed. The sensor element 9 is for example arranged in a region surrounded by the plurality of pads 21 and is fixed to the upper surface of the insulating base 19 by an adhesive. Further, not shown bonding wires are used to connect pads 41 (see FIG. 4) which are provided on the upper surface of the sensor element 9 and the plurality of pads 21. Note that, the sensor element 9 may be one mounted on the plurality of pads 21 by bumps as well.

The portion in the first channel 29 which is connected to the filter 7 and the portion in the second channel 31 which is connected to the filter 7 are separated from each other. That is, the two channels are separately connected to the filter 7. In the present embodiment, the centers in the width direction of the channels are spaced apart from each other at a periphery of the filter 7 with a distance of ¼ of the periphery.

FIG. 3B is a cross-sectional view of the sensor 1 taken along the line IIIb-IIIb in FIG. 3A.

The filter 7 is arranged so that its lower surface abuts against the upper surface of the lower layer member 13 (lower surfaces of the channels). The second channel 31 is connected to the side surface of the filter 7. This is true also for the first channel 29 which is not shown in FIG. 3B. Further, as already explained, the inflow port 3 is opened in the upper layer member 17 (opened in the upper surfaces of the channel) to expose the upper surface of the filter 7 to the outside of the package 11. Accordingly, the filter 7 does not allow the liquid specimen to pass from the upper surface to the lower surface, but allows the liquid specimen to pass from the upper surface to the side surface.

The diameter of the filter 7 is set bit larger than the diameter of the inflow port 3, and the filter 7 and the portion around the inflow port 3 of the upper layer member 17 are superimposed on each other. The double-sided tape 25 is interposed between the superimposed portions and suppresses formation of a gap between the two by adhesion to the filter 7 and upper layer member 17. That is, the double-sided tape 25 suppresses flow of the liquid specimen from the inflow port 3 to the first channel 29 or second channel 31 without passing through the filter 7.

A hydrophilic film 33 is provided on the upper surface of the lower layer member 13. The material etc. of the hydrophilic film are as explained in the description of the upper layer member 17. In the hydrophilic film 33, for example, the planar shape is made roughly the same as the planar shape of the channels (first channel 29 and second channel 31 in the present embodiment) in the package 11. The hydrophilic film 33 is for example fixed to the upper surface of the lower layer member 13 by a not shown adhesive. The position of the upper surface of the hydrophilic film 33 is suitably set by selection of the thickness of the hydrophilic film 33 and/or adjustment of the thickness of the adhesive. The position of the upper surface of the hydrophilic film 33 is made lower than the position of the upper surface of the middle layer member 15.

Accordingly, in the present embodiment, the channels are constituted between the upper layer member 17 and the hydrophilic film 33, and the hydrophilicity has become high on the upper surface and lower surface of the channels.

The height from the lower surface to the upper surface of the channels (thickness of the channels) is set relatively small. For example, the height is 50 μm to 0.5 mm. From the viewpoint of reducing the quantity of the liquid specimen (for example reducing the quantity of collection of blood), the height of the channels is preferably about 100 μm

The height of the channels is relatively small, and the contact angle with the liquid specimen on the upper surface and lower surface of the channels is small, therefore the liquid specimen can flow in the channels according to the capillary phenomenon (according to capillary force). That is, in the present embodiment, it is not necessary to make the liquid specimen flow by utilizing gravity or make the liquid specimen flow by suction from the discharge side of the channels.

That is, the first channel 29 and second channel 31 etc. are made of capillaries. The “capillaries” mean channels capable of generating a capillary phenomenon.

Note that, the capillary phenomenon may occur so far as the contact angle of the liquid at the inner surface of a channel is less than 90°. Accordingly, the inner surface of the channel only need to have a contact angle less than 90° with the liquid specimen. Further, from the viewpoint of reliably causing the capillary phenomenon, the contact angle of the inner surface of the channel with the liquid specimen is preferably less than 60°, more preferably less than 10°.

The surface having a relatively small contact angle does not have to be the entire inner surface of the channel, but may be a portion of the inner surface of the channel as well. For example, in a channel having a rectangular cross-section, the contact angle may be made relatively small only at the two side surfaces, or the contact angle may be made relatively small only at the upper and lower surfaces. In the case where the contact angle is made relatively small only in a portion of the inner surface of the channel, that portion is preferably the portion that constitutes the smallest diameter of the channel. For example, in the channel having a rectangular cross-section, in a case where the diameter between the upper and lower surfaces is smaller than the diameter between the two side surfaces, preferably the contact angle is relatively small at the upper surface or lower surface (preferably upper and lower surfaces).

The diameter of the capillary (for example the distance between the surfaces at which the contact angle is less than 90°) must be relatively small. However, it may be suitably set so far as a capillary phenomenon occurs. For example, if the diameter is 0.5 mm as explained above, a capillary phenomenon occurs well.

The capillary phenomenon can occur even in for example a groove which has an inner surface constituted by a bottom surface and two side surfaces and is opened in the upper part. Note, in the present embodiment, the capillary designates a hole or through hole (tunnel) shaped tube formed so that its inner surface of the channel surrounds the channel over 360° when viewed in a direction of flow of the liquid specimen. Further, in a capillary, unlike a cylinder meant by a tube in the narrow sense, the cross-section does not have to be circular, and the capillary does not have to be formed by a member (tube) made of a thin and long hollow rod (may be constituted by forming a hole in a package as in the embodiment).

The capillary force is stress pulling the liquid specimen in the flow direction of the capillary (penetration direction) which is generated according to a capillary phenomenon. The larger the surface tension and the smaller the contact angle and the smaller the diameter of the capillary, the larger the capillary force.

FIG. 3C is a cross-sectional view of the sensor 1 taken along the line IIIc-IIIc in FIG. 3A.

The middle layer member 15 is thicker than the sensor element 9. That is, the element arrangement portion 31b includes a space between the upper surface of the sensor element 9 and the upper layer member 17. Further, the liquid specimen flowing in the inflow portion 31a can flow onto the sensor element 9.

Also, the flow of the liquid specimen on the sensor element 9 is generated by a capillary force in the same way as that in the channel between the hydrophilic film 33 and the upper layer member 17. Note that, the contact angle of the upper surface of the sensor element 9 with the liquid specimen have only to be less than 90° in the same way as the hydrophilic film 33 etc. Preferably, it is less than 60°, more preferably less than 10°.

The hydrophilic film 33 is for example not provided in the region for arranging the sensor element 9, but is provided in the inflow portion 31a and outflow portion 31c. The position of the upper surface of the hydrophilic film 33 and the position of the upper surface of the sensor element 9 are for example schematically the same (the two upper surfaces being schematically flush). However, the upper surface positions of the portions may be set so that the upper surface on the further downstream side becomes higher.

FIG. 4 is a plan view showing the sensor element 9.

The sensor element 9 is for example constituted by a SAW (surface acoustic wave) sensor element utilizing a SAW. The sensor element 9 for example has a piezoelectric substrate 35, and a metal film 37, a pair of IDT (interdigital transducer) electrodes 39 and a plurality of pads 41 which are provided on the piezoelectric substrate 35.

The piezoelectric substrate 35 is for example constituted by a substrate of a single crystal having piezoelectricity such as a lithium tantalate (LiTaO₃) single crystal, lithium niobate (LiNbO₃) single crystal, or quartz. The planar shape and various dimensions of the piezoelectric substrate 35 may be suitably set. As an example, the thickness of the piezoelectric substrate 35 is 0.3 mm to 1.0 mm.

The metal film 37 is for example given a roughly rectangular planar shape, is located at the schematic center of the y-direction on the upper surface of the piezoelectric substrate 35, and is provided so as to be over an area which is at least equal to a detection surface 9b. The metal film 37 has for example a 2-layer structure of chromium and gold formed on the chromium. On the surface of the metal film 37, for example, an aptamer comprised of a nucleic acid or a peptide is arranged (immobilized).

The pair of IDT electrodes 39 are for generating a SAW propagated on the upper surface of the piezoelectric substrate 35 and for receiving this SAW. The pair of IDT electrodes 39 are arranged while sandwiching the metal film 37 therebetween. That is, the metal film 37 is located in the propagation path of the SAW. The line-up direction of the metal film 37 and pair of IDT electrodes 39 is for example a direction intersecting with (more specifically, perpendicular to) the second channel 31.

Each IDT electrode 39 has a pair of comb-shaped electrodes. Each comb-shaped electrode has a bus bar and a plurality of electrode fingers extending from the bus bar. Further, a pair of comb-shaped electrodes are arranged so that their plurality of electrode fingers mesh with each other. The pair of IDT electrodes 39 constitute a transversal type IDT electrode.

The frequency characteristics can be designed using the number of electrode fingers of the IDT electrode 39, the distance between adjacent electrode fingers, the crossing width of the electrode fingers, and so on as parameters. As the SAW excited by the IDT electrodes, a Rayleigh wave, Love wave, Leaky wave etc. exist. Any may be utilized as well.

An elastic member for suppressing reflection of the SAW may be provided in a region at the outside of the pair of IDT electrodes 39 in the propagation direction of the SAW as well. The frequency of the SAW can be set for example within a range of from a few megahertz (MHz) to a few gigahertz (GHz). In particular, if made several hundreds of MHz to 2 GHz, the result is practical, and reduction of size of the piezoelectric substrate 35 and consequently reduction of size of the sensor element 9 can be realized.

The plurality of pads 41 are connected to the IDT electrodes 39. The plurality of pads 41, as already explained, are connected through not shown bonding wires to the plurality of pads 21 on the lower layer member 13. Further, signals input from the terminals 5 are input through the pads 21 and 41 to the IDT electrodes 39. Further, the signals output from the IDT electrodes 39 are output through the pads 41 and 21 to the terminals 5.

The IDT electrodes 39, pads 41, and lines connecting them are for example made of gold, aluminum, alloys comprised of aluminum and copper, or the like. These conductors may be given a multi-layer structure as well. In the case of a multi-layer structure, for example, the first layer is made of titanium or chromium, the second layer is made of aluminum, aluminum alloy, or gold, and further titanium or chromium may be laminated as the topmost layer. Note that, the thicknesses of these conductors are for example less than 1 μm, therefore the influence of these exerted upon the height of the second channel 31 (for example 50 μm or more) is small.

When the liquid specimen comes into contact with the metal film 37 on which the aptamer is arranged, a specific target substance in the liquid specimen is bound to the aptamer corresponding to that target substance, therefore the weight of the metal film 37 changes. As a result, the phase characteristic etc. of the SAW propagated between a pair of IDT electrodes 39 change. Accordingly, the property or ingredient of the liquid specimen can be checked based on the change of the phase characteristic and so on.

Note that, in the upper surface of the sensor element 9, mainly the region between the pair of IDT electrodes 39 becomes the detection surface 9b on which the liquid specimen should be arranged when measuring an ingredient of the liquid specimen. It is not always necessary to arrange the liquid specimen in regions other than the detection surface 9b. Note, from the viewpoint of reducing the variation of test results by always making the quantity of the liquid specimen which is arranged on the detection surface 9b constant, preferably the liquid specimen is filled in the element arrangement portion 31b.

(Procedure of Test Method)

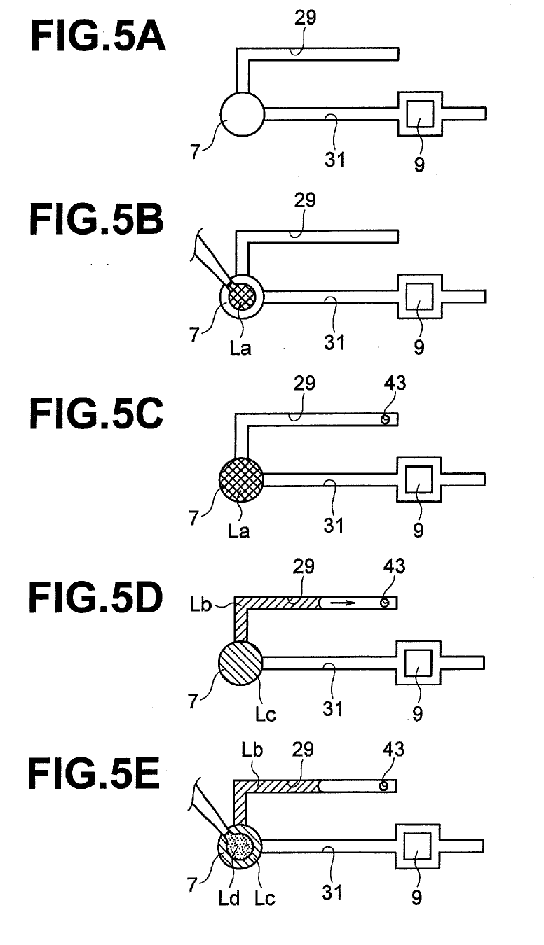

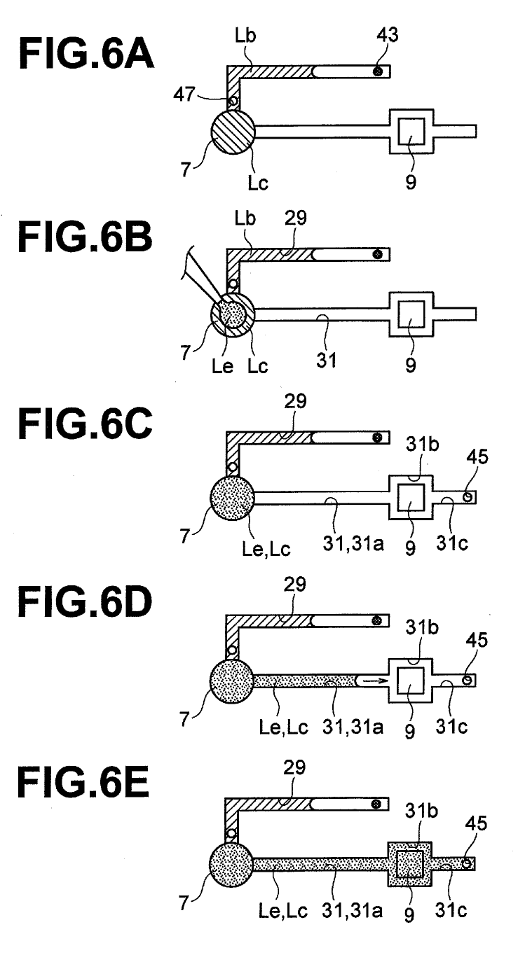

FIG. 5A to FIG. 5E and FIG. 6A to FIG. 6E are diagrams for explaining procedures of the test method of a liquid specimen using the sensor 1. These diagrams diagrammatically show the planar shapes etc. of the filter 7, first channel 29, and second channel 31.

FIG. 5A shows a state where the liquid specimen has not yet been supplied to the sensor 1. At this time, in the sensor 1, holes have not yet been formed at the positions indicated by the marks 6.

Next, as shown in FIG. 5B, the liquid specimen La is supplied through the inflow port 3 to the filter 7. For the supply of the liquid specimen La to the filter 7, usage of a dropper or injector or another known suitable method may be employed. When the liquid specimen La is supplied to the inflow port 3, for example, in the liquid specimen La, (a part or all of) the ingredient capable of passing through the filter 7 are guided into the holes of the filter 7 due to the capillary force of the holes of the filter 7, and the remainder stays on the filter 7.

The liquid specimen La is for example blood diluted by PBS (phosphate buffered saline). Note that, the PBS and blood may be mixed before supply to the filter 7 or may be mixed inside and/or on the filter 7 by previously supplying one to the filter 7 and supplying the other to the filter 7 later.

Preferably, after supplying the PBS to the filter 7, the blood is supplied to the filter 7 and the PBS and blood are mixed inside and/or on the filter 7. By doing this, it becomes unnecessary to perform a dilution of the blood separately from the work for the sensor 1, therefore the instrument and work are simplified.

Next, as shown in FIG. 5C, a first hole 43 for communicating a predetermined portion (first portion) in the flow direction in the first channel 29 with an external portion of the package 11 is opened. The first portion is preferably a portion downstream from the intermediate position of the first channel 29, more preferably the downstream end part of the first channel 29. The first mark 6A shown in FIG. 1 indicates the position at which the first hole 43 should be opened, and is of some help for the user when opening the first hole 43 by manual labor.

Note that, the first hole 43 may be formed by the user by manual labor or may be automatically formed by the reader 103 which will be explained later. Formation of the first hole 43 may be suitably performed by apparatuses publicly known. For example, the first hole 43 may be formed by a needle or cutting tool (drill etc.). Also, for example, the first hole 43 may be formed by removing a portion of the upper layer member 17 by heat by an abutment of a heated pin or irradiation of a laser beam. The upper layer member 17 may be constituted so that the first hole 43 is easily formed. For example, The upper layer member 17 is formed thinly at the position where the first hole 43 is to be formed. The same is also true for the second hole 45 and third hole 47 which will be explained later.

As shown in FIG. 5D, by opening the first hole 43, the gas (for example air) in the first channel 29 can escape to the outside of the package 11. As a result, an ingredient in the liquid specimen La which can pass through the filter 7 (non-tested object ingredient Lb, for example blood plasma (and PBS)) flows from the filter 7 into the first channel 29 due to the capillary force. On the other hand, an ingredient as a tested object of the sensor 1 (tested object ingredient Lc, for example blood cells) cannot pass through the filter 7, therefore remains on the filter 7. In this way, in the liquid specimen La, the non-tested object ingredient Lb is separated from the tested object ingredient Lc and does not flow into the sensor element 9, but is retracted to the first channel 29.

Note that, the supply of the liquid specimen La in FIG. 5B and the formation of the first hole 43 in FIG. 5C may be carried out in an order reverse to that described above. In any case, as shown in FIG. 5D, the non-tested object ingredient Lb passed through the filter 7 flows into the first channel 29, and the tested object ingredient Lc remains on the filter 7. Further, in a case where the liquid specimen La is a diluted one, and a liquid specimen in a narrow sense (for example blood) before dilution and a solvent for diluting this (buffer solution, for example PBS) are separately supplied to the filter 7, the first hole 43 may be formed after supplying one to the filter 7, then the other may be supplied to the filter 7.

After that, when flow of the non-tested object ingredient Lb to the first channel 29 is stopped, as shown in FIG. 5E, rinse water Ld is supplied through the inflow port 3 to the filter 7. For example, when the liquid specimen La contains blood, the PBS is supplied as the rinse water Ld. Due to this, the non-tested object ingredient Lb remaining on the filter 7 can be made to flow into the first channel 29 together with the rinse water Ld.

Note that, stopping of flow of the non-tested object ingredient Lb to the first channel 29 may be judged by a suitable method. For example, the judgment may be carried out visually through a transparent upper layer member 17 or may be carried out according to whether a predetermined time has passed. Further, the supply of the rinse water Ld may be omitted as well.

Next, as shown in FIG. 6A, the first hole 43 is closed. Note that, the blockade of the first hole 43 may be carried out by the user by manual labor or may be automatically carried out by the reader 103 explained later. The blockade of the first hole 43 may be carried out by for example applying a finger or member to the hole, adhering a seal, filling an adhesive, or the like.

When the first hole 43 is closed, the gas loses its escape route at the downstream side portion of the first channel 29, therefore inflow of the liquid to be supplied to the filter 7 after that to the first channel 29 is suppressed. Further, in the downstream side portion of the first channel 29, the gas is no longer supplemented, therefore backward flow (flow to the filter 7) of the liquid (non-tested object ingredient Lb) which has been already held in the first channel 29 is also suppressed.

Further, in the step of FIG. 6A, a third hole 47 for communicating a predetermined portion (second portion) in the first channel 29 upstream from the portion (first portion) at which the first hole 43 is provided with the external portion of the package 11 is opened. The second portion is preferably an upstream side portion from an intermediate position in the first channel 29, more preferably the upstream end part of the first channel 29. The third mark 6C shown in FIG. 1 indicates the position at which the third hole 47 should be opened, and is of some help for the user when opening the third hole 47 by manual labor.

The formation of the third hole 47 means a break at the upper surface of the first channel 29 to be wetted by the liquid, consequently it becomes hard for the liquid to ride over the position of the third hole 47 by the capillary phenomenon. As a result, inflow of the liquid which is supplied to the filter 7 after that to the first channel 29 is suppressed. Further, backward flow (flow to the filter 7) of the liquid (non-tested object ingredient Lb) which has been already held in the first channel 29 is suppressed.

Next, as shown in FIG. 6B, a liquid Le for dissolving the tested object ingredient Lc (liquid reacting with the liquid specimen) is supplied through the inflow port 3 to the filter 7. In the case where the tested object ingredient Lc is red blood cells, the liquid Le is for example purified water (H₂O).

The tested object ingredient Lc can pass through the filter 7 by dissolution in the liquid Le. Further, the tested object ingredient Lc is guided to the hole of the filter 7 by capillary force. Note, as explained above, inflow of the liquid Le in which the tested object ingredient Lc is dissolved to the first channel 29 is suppressed.

Next, as shown in FIG. 6C, the second hole 45 which communicates a predetermined position (for example downstream end part) in the second channel 31 downstream (outflow portion 31c) from the sensor element 9 with an external portion of the package 11 is opened. The second mark 6B shown in FIG. 1 indicates the position at which the second hole 45 should be opened, and is of some help for the user when opening the second hole 45 by manual labor.

As shown in FIG. 6D, by opening the second hole 45, the gas in the second channel 31 can escape to the outside of the package 11. As a result, the liquid Le dissolving the tested object ingredient Lc therein flows from the filter 7 into the second channel 31 due to capillary force. Note that, inflow (backward flow) of the non-tested object ingredient Lb which is already held in the first channel 29 to the second channel 31 is suppressed as explained above.

Note that, the supply of the liquid Le in FIG. 6B and the formation of the second hole 45 in FIG. 6C may be carried out in an order reverse to that described above as well. In any case, as shown in FIG. 6D, the liquid Le dissolving the tested object ingredient Lc therein flows into the second channel 31.

After that, as shown in FIG. 6E, the liquid Le dissolving the tested object ingredient Lc therein is filled in the element arrangement portion 31b. Note that, by provision of the outflow portion 31c and provision of the second hole 45 in the outflow portion 31c, the liquid Le passes through the element arrangement portion 31b and tries to further flow into the outflow portion 31c. That is, the outflow portion 31c facilitates filling of the element arrangement portion 31b with the liquid Le.

In this way, by arrangement of the liquid Le dissolving the tested object ingredient Lc on the upper surface of the sensor element 9, it becomes possible to measure the composition of the tested object ingredient Lc by the sensor element 9. Note that, the sensor element 9 may be mounted onto the reader before, after, or in the middle of the above procedures explained with reference to FIG. 5A to FIG. 6E.

(Modifications of Sensor)

The sensor explained above and the testing method using the sensor can be modified in various ways. In the following description, these modifications will be listed.

Either of the blockade of the first hole 43 or formation of the third hole 47 in FIG. 6A may be omitted as well. The inflow to the first channel 29 and the backward flow thereof are suppressed even if only one is carried out.

Further, both of blockade of the first hole 43 and formation of the third hole 47 may be omitted as well. For example, at the time when the liquid Le for dissolving the tested object ingredient Lc is supplied (FIG. 6B), even if the liquid Le flows into the first channel 29, so far as the liquid Le flows also into the second channel 31, testing of the tested object ingredient Lc is possible although there is the inconvenience of increased waste of the tested object ingredient Lc. Further, for example, if the second hole 45 is formed after supplying the liquid Le to the filter 7, although the non-tested object ingredient Lb flows into the filter 7 from the first channel 29, the tested object ingredient Lc which has been already supplied to the filter 7 flows into the second channel 31 prior to the non-tested object ingredient Lb which flows into the filter 7, therefore the tested object ingredient Lc can be tested.

The first hole 43 may be provided in the sensor 1 from the first as well. In other words, the first hole 43 may be provided not by the user of the sensor 1, but by the manufacturer of the sensor 1. As already explained, this is because the first hole 43 may be provided before the supply of the liquid specimen La in FIG. 5B as well.

Further, in addition to the first hole 43, the second hole 45 and third hole 47 may also be provided by the manufacturer of the sensor 1. In this case, the third hole 47 is blockade at the steps in FIG. 5A to FIG. 5E, and the blockade is released at the step in FIG. 6A and on. In the same way, the second hole 45 is blockade at the steps in FIG. 5A to FIG. 6B, and the blockade is released at the step in FIG. 6C and on.

Note that, the blockades of the holes and release thereof may be carried out by the user by manual labor or may be automatically carried out by the reader 103 which will be explained later. Further, the blockades may be carried out by for example applying a finger or member as alluded to in the explanation of FIG. 6A. Note that, for example, the state where the seals closing the holes in the sensor 1 are peeled off may be grasped as formation of holes in a sensor not provided with holes or may be grasped as release of blockades of holes in a sensor provided with holes.

Below, the case where all holes (two, ie., 43 and 45 or three, i.e., 43, 44, and 45) were formed by the user, the case where only the first hole 43 was formed by the manufacturer and the remaining holes were formed by the user, and the case where all holes were formed by the manufacturer were exemplified. Other than these, the following various combination may be considered: Either one or two among the three holes (43, 45, and 47) or either one of the two holes (43 and 45) is provided by the manufacturer of the sensor 1 and the remainder are provided by the user. Any of them may be employed as well.

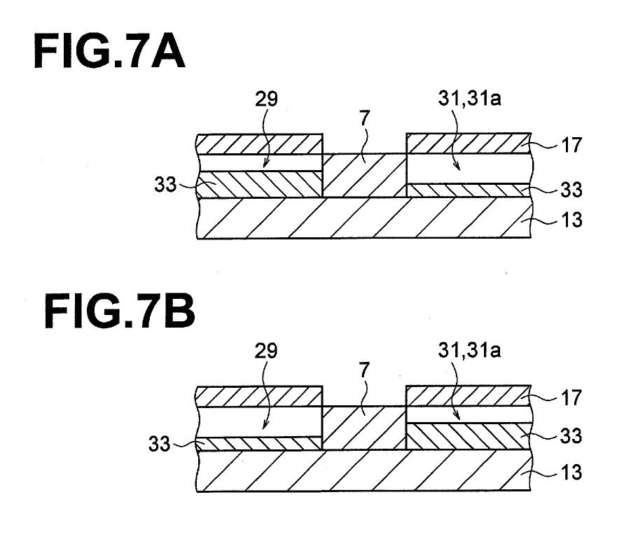

FIG. 7A and FIG. 7B are cross-sectional views showing examples in which the capillary forces of the first channel 29 and the second channel 31 are made different from each other.

In FIG. 7A, the capillary force of the first channel 29 is made larger than the capillary force of the second channel 31. Specifically, the height from the lower surface to the upper surface of the first channel 29 is made smaller than the height from the lower surface to the upper surface of the second channel 31. From another viewpoint, the cross-sectional area of the first channel 29 is made smaller than the cross-sectional area of the second channel 31 (inflow portion 31a).

Conversely, in FIG. 7B, the capillary force of the second channel 31 is made larger than the capillary force of the first channel 29. Specifically, the height from the lower surface to the upper surface of the second channel 31 is made smaller than the height from the lower surface to the upper surface of the first channel 29. From another viewpoint, the cross-sectional area of the second channel 31 (inflow portion 31a) is made smaller than the cross-sectional area of the first channel 29.

Such setting of heights of the channels is realized by making the position of the upper surface of the hydrophilic film 33 different from each other between the first channel 29 and the second channel 31. Note that, the position of the upper surface of the hydrophilic film 33, as already explained, can be adjusted according to the thickness of the adhesive for adhering the hydrophilic film 33 to the lower layer member 13 other than the thickness of the hydrophilic film 33.

As shown in FIG. 7A, when the capillary force of the first channel 29 is made larger than the capillary force of the second channel 31, it is hard to cause the flow from the first channel 29 to the second channel 31. Accordingly, for example, when having the second hole 45 communicated (forming a hole or release of a blockade) (FIG. 6C), flow of the non-tested object ingredient Lb which is held in the first channel 29 into the second channel 31 is suppressed. As a result, for example, mixing of the non-tested object ingredient Lb into the tested object ingredient Lc is suppressed, therefore the test precision is improved.

As shown in FIG. 7B, when the capillary force of the second channel 31 is made larger than the capillary force of the first channel 29, the flow to the second channel 31 occurs more easily. Accordingly, for example, flow of the liquid Le for dissolving the tested object ingredient Lc into the first channel 29 is suppressed, therefore the tested object ingredient Lc can be made to flow into the second channel 31 without waste. As a result, for example, the quantity of the liquid specimen is reduced, and consequently the load of the user is lightened. Further, the cross-sectional area of the first channel 29 is easily made larger, therefore it becomes easier to secure the capacity for holding the non-tested object ingredient Lb.

In this way, each of the examples in FIG. 7A and FIG. 7B has merits. In actual products, either example may be suitably selected in accordance with the intended use of the sensor 1 or other flow control factors of the sensor 1 (presence/absence of the blockade of the first hole 43 and communication (formation of hole or release of the blockade) of the third hole 47). Also, the capillary force of the first channel 29 and the capillary force of the second channel 31 may be the same degree as each other as well.

Note that, as the method of making the capillary forces of the first channel 29 and the second channel 31 different from each other, the case where the heights of the two channels were made different from each other was exemplified. However, the capillary forces of the two channels may be different from each other by making the materials forming the inner surfaces of the two channels (hydrophilicities) different from each other as well. For example, the hydrophilic film 33 of the first channel 29 and the hydrophilic film 33 of the second channel 31 (inflow portion 31a) may be made of materials which are different from each other as well. Note that, the materials different from each other referred to here include materials obtained by hydrophilizing the same base materials to extents different from each other and thereby making the hydrophilicities different from each other.

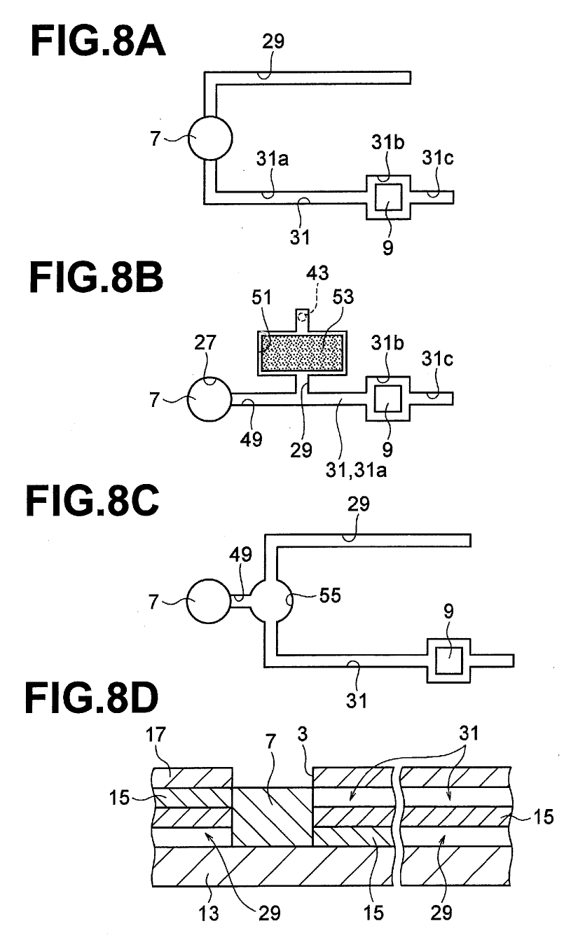

FIG. 8A to FIG. 8D and FIG. 9A to FIG. 9C and FIG. 11 show various modifications of shapes of the first channel 29 and second channel 31. Specifically, they are as follows.

In the example in FIG. 8A, the portion of the first channel 29 which is connected to the filter 7 and the portion of the second channel 31 which is connected to the filter 7 extend from positions opposite to each other to directions opposite to each other with respect to the filter 7.

In this case, the two channels are separated to the maximum, and the inflowing directions due to the capillary forces of the two channels are inverse in orientation to each other. Therefore, for example, flow of the non-tested object ingredient Lb held in the first channel 29 into the second channel 31, flow of the tested object ingredient Lc into the first channel 29, and/or mixing of the two ingredients are suppressed.

Note that, unlike this example, in a case where the first channel 29 and the second channel 31 extend to directions crossing each other, for example, since the two channels do not extend to opposite directions to each other, it becomes possible to make the width of the sensor 1 narrower. Further, for example, since the flow directions of the channels do not coincide, when a backward flow is generated in one channel, inflow to the other channel is suppressed.

In the example in FIG. 8B, the first channel 29 and the second channel 31 do not extend from the filter 7 (are not directly connected to the filter 7), but extend from a common channel 49 connected to the filter 7 (specifically, for example, from the downstream end part of the common channel 49) to directions different from each other (so as to be separated from each other). From another viewpoint, a portion of the channel (common channel 49) is shared by the non-tested object ingredient Lb and the tested object ingredient Lc.

In this case, for example, the area of the channels as a whole can be made smaller while arranging the sensor element 9 and/or retreat chamber 51 which will be explained later at any position in the package 11. In this example, the filter 7 is not interposed between the first channel 29 and the second channel 31, therefore the effects according to the difference of capillary forces between the two channels which was explained with reference to FIGS. 7A and 7B become larger.

Note that, conversely to this example, in a case where the first channel 29 and the second channel 31 extend from the filter 7, the channels are completely separated between the non-tested object ingredient Lb and the tested object ingredient Lc (except the filter 7), therefore mixing of the two ingredients is suppressed.

Further, in the example in FIG. 8B, the first channel 29 has the retreat chamber 51. The retreat chamber 51 is formed by the cross-sectional area (more specifically, width) being made larger in a portion of the first channel 29, and the cross-sectional area being larger than those of the inlet and outlet thereof. Preferably, the cross-sectional area of the retreat chamber 51 is larger than the cross-sectional area of the inlet through which the liquid specimen flows to the first channel 29 (in the example in FIG. 8B, the cross-section of the opening of the first channel 29 with respect to the common channel 49). The planar shape of the retreat chamber 51 may be suitably set, but is for example rectangular.

By the provision of the retreat chamber 51, the capacity for holding the non-tested object ingredient Lb is easily secured without increase of size of the sensor 1.

Note that, in the same way as the provision of the outflow portion 31c for suitably filling the element arrangement portion 31b with the tested object ingredient Lc (and liquid Le), downstream from the retreat chamber 51, a portion having a smaller cross-sectional area than that of the retreat chamber 51 is formed. The first hole 43 is provided in this portion. Note, such a portion having a small cross-sectional area need not be provided (the outlet of the retreat chamber 51 need not be formed). In this case, for example, the first hole 43 may be provided at a suitable position on downstream side in the retreat chamber 51. Further, the inlet of the retreat chamber 51 may be opened in the common channel 49 (filter arrangement portion 7 in a case where the common channel 49 is not provided) without interposition of the channel.

Further, in the example in FIG. 8B, the sensor 1 has a porous material 53 arranged in the first channel 29. The porous material 53 is for example made of ceramic or sponge. It may be constituted by the same material as that for the filter 7. The porous material 53 may be arranged at a suitable position in the first channel 29 having a suitable shape and is for example arranged in the retreat chamber 51.

The porous material 53 is relatively small in pore size (at least smaller than the diameter of the portion of the first channel 29 in which the porous material 53 is to be arranged), therefore has a large capillary force. Accordingly, by holding the non-tested object ingredient Lb in the holes of the porous material 53, the backward flow (flow to the second channel 31) of the non-tested object ingredient Lb can be suppressed. Further, a capillary force sucking the non-tested object ingredient Lb is generated by the porous material 53. Therefore, when the porous material 53 is provided in the retreat chamber 51, the necessity of generating the capillary force by the retreat chamber 51 itself becomes low. As a result, for example, in the retreat chamber 51, not only the width, but also the height can be made large.

Note that, as understood from the description of the action of the porous material 53 described above, the pore sizes of the porous material 53 do not have to be within a relatively narrow range (be even) unlike the pore sizes of the filter 7. The cross-sectional area of the holes in the porous material 53 is preferably small from the viewpoint of generating a large capillary force, but preferably large from a viewpoint of keeping a large quantity of non-tested object ingredient Lb. For example, a mean value of the cross-sectional areas of holes in the porous material 53 is smaller than that in the narrowest portion in the first channel 29, but is larger than the cross-sectional areas of the holes in the filter 7.

The porous material 53 may be arranged over the entire first channel 29 as well. In this case, it may be grasped that the first channel itself is constituted by a porous material.

In the example in FIG. 8C, downstream of the filter 7, provision is made of a mixing chamber 55 for mixing the non-tested object ingredient Lb and the solvent (buffer solution) and/or mixing the tested object ingredient Lc and the liquid Le for dissolving this.

The mixing chamber 55 is for example positioned at a diverging point of the common channel 49, the first channel 29, and the second channel 31. From another viewpoint, the common channel 49 has the mixing chamber 55 in the downstream end part. From still another viewpoint, the mixing chamber 55 is positioned between the filter 7 and the first channel 29 and is positioned between the filter 7 and the second channel 31. The portion of the first channel 29 which is connected to the common channel 49 and the portion of the second channel 31 which is connected to the common channel 49 are separated from each other.

In the mixing chamber 55, the cross-sectional area (more specifically, width) is made larger than those of its inlet and outlet. Preferably, the cross-sectional area of the mixing chamber 55 is larger than the cross-sectional area of the inlet through which the liquid specimen flows into the common channel 49 (in the example in FIG. 8C, opening area of the common channel 49 with respect to the filter 7). The planar shape of the mixing chamber 55 may be suitably set and is for example circular.

The mixing chamber 55 is made larger in cross-sectional area than those of its inlet and outlet, therefore the liquid flowing into the mixing chamber 55 stays in the mixing chamber 55, and a flow that circulates in the mixing chamber 55 is apt to be generated.

Accordingly, for example, when the non-tested object ingredient Lb and the solvent (buffer solution) are supplied in order (either may be first) to the filter 7, they are suitably mixed. As a result, for example, the non-tested object ingredient Lb having a high viscosity is suitably diluted, so the non-tested object ingredient Lb smoothly flows into the first channel 29.

Further, for example, when the liquid Le which dissolves the tested object ingredient Lc is supplied to the filter 7, the dissolved tested object ingredient Lc and the liquid Le are suitably mixed. As a result, for example, with respect to the concentration of the tested object ingredient Lc which is arranged on the sensor element 9, variation among a plurality of tests or variation on the sensor element 9 is reduced.

Note that, the mixing chamber 55 was provided at the diverging point in FIG. 8C, but may be provided in the middle of the common channel 49 as well. Further, the mixing chamber 55 may be provided in the first channel 29 only for the purpose of mixing the non-tested object ingredient Lb and the solvent (buffer solution) as well. Note that, in this case, the common channel 49 is unnecessary, and the mixing chamber 55 is preferably provided on the upstream side from the intermediate position of the first channel 29. Further, the mixing chamber 55 may be provided in the second channel 31 only for the purpose of mixing the tested object ingredient Lc and the liquid Le for dissolving this as well. Note that, in this case, the common channel 49 is unnecessary, and the mixing chamber 55 is provided on the upstream side from the sensor element.

In the example in FIG. 8D, at least a portion of the first channel 29 and at least a portion of the second channel 31 are different from each other in the positions in the thickness direction of the channels (in the example in FIG. 8D, the direction of stacking of the layer shaped members constituting the package 11).

Specifically, for example, in the portion connected to the filter 7, the first channel 29 is positioned at a lower position than the second channel 31 (on the opposite side to the inflow port 3). Further, at a suitable position, the first channel 29 and the second channel 31 are superimposed on each other. For example, the two channels cross each other at different levels.

By the positions of the first channel 29 and the second channel 31 in the vertical direction being different from each other in the vicinity of the filter 7, mixing of the liquids which should flow to the two channels is suppressed. In particular, in a case of a positional relationship where the first channel 29 is separated from the inflow port 3 more than the second channel 31, when supplying the liquid Le for dissolving the tested object ingredient Lc to the inflow port 3, the liquid Le previously reaches the second channel 31, and it is hard for the non-tested object ingredient Lb in the first channel 29 to flow backward against the flow of the liquid Le, therefore mixing is suppressed. In a case where the inflow port 3 is opened in the upper part (here, upper part using gravity as the standard), gravity can be utilized for preventing backward flow of the non-tested object ingredient Lb, therefore this is more preferred.

Further, in this example, by superimposing the first channel 29 and the second channel 31 on each other at a suitable positions, it is easy to secure the capacity of the first channel 29 while making the area of the sensor 1 small. Further, also the degree of freedom of design for the shapes of the channels is improved.

The first channel 29 and the second channel 31 which are different from each other in positions in the vertical direction in at least a portion in this way may be suitably realized. FIG. 8D exemplifies a case where this is realized by stacking of three middle layer members 15 between the lower layer member 13 and the upper layer member 17 and formation of penetration grooves in the middle layer members 15 on the two sides among them. Note that, in this case, the middle layer member 15 at the center may be constituted by a hydrophilic film in the same way as the upper layer member 17.

Other than this, for example, the channel constitution as in FIG. 8D may be realized by providing only one middle layer member 15 between the lower layer member 13 and the upper layer member 17 and forming concave grooves (bottomed grooves) in the two major surfaces of the middle layer member 15 and forming a through hole.

Note that, either of the first channel 29 or second channel 31 may be located below the other over the entire channels as well. Further, in the first channel 29 and the second channel 31, although parts are made different from the other in the vertical direction, another part of the first channel 29 (for example retreat chamber 51) or another part of the second channel 31 may have a height reaching the upper layer member 17 from the lower layer member 13 as well.

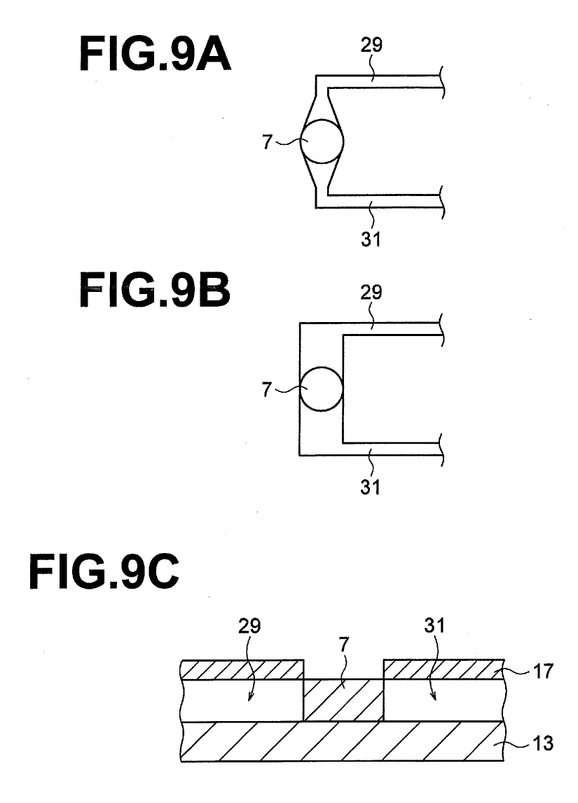

In the examples in FIG. 9A and FIG. 9B, in the channels extending from the filter 7 (in the examples in these diagrams, the first channel 29 and second channel 31. They may be the common channel 49 (FIG. 8B and FIG. 8C) as well), the widths of the parts thereof connected to the filter 7 become broader than the widths of the downstream side portions thereof when viewed in the stacking direction of the layer shaped members of the package 11 (vertical direction). More specifically, for example, the widths of the connection parts become widths equal to the width of the filter 7.

In the example in FIG. 9A, the parts connected to the filter 7 gradually become smaller in width and are smoothly connected to the downstream side parts. Further, in the example in FIG. 9A, the parts connected to the filter 7 have a constant width, but suddenly change in width at the borders with the downstream side parts.

In such a constitution, for example, in the same way as the mixing chamber 55, mixing of the liquids can be promoted downstream of the filter 7 while smoothly making the liquid flow out of the filter 7 to a channel (first channel 29 or second channel 31 in the example in the diagrams). From the viewpoint of making the liquid smoothly flow to the downstream side, the example in FIG. 9A is preferred, while from the viewpoint of making the stagnation/mixing action of the liquid large, the example in FIG. 9B is preferred.

Note that, unlike the examples in FIG. 9A and FIG. 9B, in a case as shown in FIG. 3A where the cross-sectional areas (heights and/or widths) of the portions of the channels which are connected to the filter 7 are smaller than the cross-sectional area of the filter 7 when viewed in the flow directions of the portions, stagnation/mixing in the filter 7 is promoted. Either may be suitably selected in accordance with various conditions such as the type of liquid specimen and size of the sensor 1.

In the example in FIG. 9C, the channels extending from the filter 7 (in the example in this diagram, they are the first channel 29 and second channel 31, but may be the common channel 49 (FIG. 8B and FIG. 8C) as well) are formed so that the lower surfaces of the channels and the lower surface of the filter 7 roughly are flush when viewed in a direction perpendicular to the stacking direction of the layer shaped members of the package 11. From another viewpoint, in this example, when viewed in a direction perpendicular to the stacking direction of the layer shaped members of the package 11, the heights from the lower surfaces to the upper surfaces of the channels become equal to the thickness of the filter 7.

Such a constitution is realized by for example arranging the filter 7 on the upper surface of the lower layer member 13 and constituting the lower surfaces of the channels by the upper surface of the lower layer member 13. Note that, in the upper surface of the lower layer member 13, preferably at least regions constituting the lower layers of the channels are hydrophilized.

In the constitution in this example, the liquid supplied to the filter 7 smoothly flows into the channels.

Note that, unlike the example in FIG. 9C, in the case as in FIG. 3B where the lower surface of the portion of the channel which is connected to the filter 7 (in the example in FIG. 3B, the upper surface of the hydrophilic film 33) is positioned on the side closer to the upper surface side of the channel than the lower surface of the filter 7, the liquid stagnates in the filter 7 and mixing is promoted.

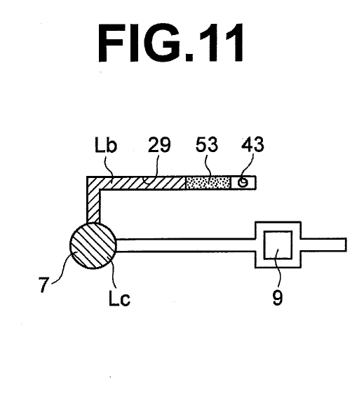

In the example in FIG. 11, in the same way as the example in FIG. 8B, the porous material 53 is provided in the first channel 29. Note, in this example, the porous material 53 is provided not in the retreat chamber 51 by interposition of the common channel 49, but in an ordinary channel portion (for example a portion of the channel having a constant cross-sectional area).

According to such a constitution, when the liquid specimen (non-tested object ingredient Lb) reaches the porous material 53, the non-tested object ingredient Lb is pulled to the downstream side by the relatively large capillary force of the porous material 53. Accordingly, for example, the backward flow of the non-tested object ingredient Lb to the filter 7 side is reduced. As a result, for example, the blockade of the first hole 43 and/or communication of the third hole 47 explained with reference to FIG. 6A can be made unnecessary. Naturally, the porous material 53 may be used together with the blockade of the first hole 43 and/or communication of the third hole 47.

Note that, the porous material 53 for example can be formed so as to have a cross-sectional area equal to the cross-sectional area of the first channel 29 and have a suitable length shorter than the first channel 29. Further, the porous material 53, for example, may be positioned on the downstream side from the intermediate position of the first channel 29 or may be arranged so that the downstream side end part is adjacent to the first hole 43.

FIG. 10A is a perspective view showing a unit 101 having the sensor 1 and the reader 103 to/from which the sensor 1 is attached/detached.

As already explained, the sensor 1 is held in the reader 103 and is electrically connected to the reader 103 and used by inserting the portion on the terminals 5 side into the slot of the reader 103. In the example in FIG. 10A, the inflow port 3 is exposed to the outside of the reader 103 in the mounted state so that the liquid specimen can be supplied to the inflow port 3 after mounting the sensor 1 on the reader 103.

The reader 103 is for example connected to a PC (personal computer) 105 for use. The PC 101 for example makes a display portion display information prompting the operation of the user and outputs a control signal to the reader 103 based on the operation of the user with respect to the control unit. The reader 103 inputs the electrical signal to the sensor 1 according to the control signal from the PC 101. Further, the reader 103 performs suitable processing with respect to the electrical signal output from the sensor 1, for example, amplification, filtering, or AD conversion, and outputs the electrical signal after that processing to the PC 101. The PC 101 makes the display portion display the information of the property or ingredient of the liquid specimen based on the electrical signal from the reader 103.

FIG. 10B and FIG. 10C are block diagrams showing the constitutions of a signal processing system of the reader 103. FIG. 10B shows an example of a case where the reader 103 is constituted so as to be able to form the first hole 43, second hole 45, and third hole 47 in the sensor 1. FIG. 10C shows an example of a case where the reader 103 is constituted so as to be able to close the first hole 43, second hole 45, and third hole 47 which are formed in the sensor 1 in advance.

As shown in these diagrams, the reader 103 has a transmitting circuit 107 which generates an electrical signal to be input to the sensor 1, a receiving circuit 109 which receives the electrical signal output from the sensor 1, a control part 111 which controls them, and a power supply unit 113 for supplying power to them.

The transmitting circuit 107 is for example constituted by an IC or the like and includes a high frequency circuit. Further, the transmitting circuit 107 generates an AC signal having a frequency and voltage in accordance with the signal from the control part 111 and inputs the same to the sensor 1.

The receiving circuit 109 is for example constituted by an IC or the like and includes an amplification circuit, filter, or AD conversion circuit. Further, the receiving circuit 109 applies suitable processing to the electrical signal output from the sensor 1 and outputs the result to the control part 111.

The control part 111 is constituted by including a CPU, ROM, RAM, etc. Further, it drives the transmitting circuit 107 and receiving circuit 109 based on the control signal from the PC 101.

The power supply unit 113 is constituted including an inverter or converter and converts electric power from a commercial power supply or the PC 101 to a suitable voltage and applies the voltage after the conversion to the transmitting circuit 107, receiving circuit 109, and control part 111.

The reader 103 in the example in FIG. 10B further has a first hole forming member 115A, second hole forming member 115B, and third hole forming member 115C for forming the first hole 43, second hole 45, and third hole 47 (below, sometimes they will be simply referred to as the “hole forming members 115” and will not be discriminated).

The hole forming members 115 are for example needles, cutting tools, heated pins or portions of lasers emitting laser beams. The hole forming members 115 are arranged at positions capable of communicating the holes. For example, the hole forming members 115 are positioned right above the positions at which the holes should be formed.

All of the operations of the hole forming members 115 may be automatically carried out, a portion may be automatically carried out, or all of them may be carried out by the operation of the user. For example, all of the operations of forming three holes in order may be automatically carried out based on the control of the control part 111 triggered by a predetermined operation of the user with respect to the reader 103 or PC 105. Further, for example, the user may perform the operation of starting the formation of each hole with respect to the reader 103 or PC 105 for the three holes in order, and each hole forming member 115 may be driven under the control of the control part 111 whenever that instruction is carried out. Further, for example, needles or cutting tools may be formed so that they can be manually moved by the user, and holes may be formed by direct operation of the user with respect to these hole forming members 115.

The reader 103 in the example in FIG. 10C, unlike the example in FIG. 10B, has a first blockade member 117A, second blockade member 117B, and third blockade member 117C for closing the first hole 43, second hole 45, and third hole 47 (below, sometimes they will be simply referred to as the “blockade members 117” and will not be discriminated).

The blockade members 117 are for example members for closing holes by abutting against the upper surface of the sensor 1 so as to cover the holes. In the blockade members 117, preferably at least the portions abutting against the upper surface of the sensor 1 are constituted by elastic members (rubber) in order to improve the sealability. The blockade members 117 are arranged at the positions where they can cover the holes. For example, the blockade members 117 are positioned right above the positions at which the holes should be formed. Further, the blockade members 117 are movable in the vertical direction.

The blockade members may be moved by a drive unit such as electric motor or electromagnet or may be moved by a direct operation by the user. Note that, in a case where they are moved by the drive unit, the movement of the blockade members may be wholly automatically carried out or a portion may be automatically carried out. That is, all of the operations of closing the three holes in a suitable order may be automatically carried out based on the control of the control part 111 triggered by a predetermined operation of the user with respect to the reader 103 or PC 101, also, for example, the user may instruct the blockade or release of it for each of holes with respect to the reader 103 or PC 105 in a suitable order.

Note that, FIG. 10B shows the reader 103 in the case where all of three holes (43, 45, and 47) are formed by the user, and FIG. 10C shows the reader 103 in the case where all of the three holes were formed by the manufacturer and are closed by the user. However, when these two readers 103 are suitably combined, it is apparent that sensors and test methods in various aspects concerning the formation and utilization of three holes which were already explained can be coped with.

For example, concerning the sensor 1 and test method in an aspect explained with reference to FIG. 5 and FIG. 6 in which all of the three holes are formed by the user and the first hole 43 is closed after holding the non-tested object ingredient Lb in the first channel 29, it is enough to add the first blockade member 117A in FIG. 10C to the reader 103 having three hole forming members 115 in FIG. 10B. Note that, in this case, either or both of the first hole forming member 115A and first blockade member 117A may be constituted so as to stand by slanted upward relative to the first hole 43 and move at a slant relative to the vertical direction.

Further, for example, concerning the sensor 1 and testing method in an aspect where the first hole 43 has been provided by the manufacturer, the second hole 45 and third hole 47 are provided by the user, and the first hole 43 is closed after holding the non-tested object ingredient Lb in the first channel 29, it is enough to provide the reader 103 having three hole forming members 115 in FIG. 10B with the first blockade member 117A in FIG. 10C in place of the first hole forming member 115A.

Note that, in embodiment or modifications explained above, the sensor 1 is one example of the liquid specimen sensor, the portion at which the first mark 6A and/or first hole 43 is provided is one example of the first hole portion, the portion at which the second mark 6B and/or second hole 45 is provided is one example of the second hole portion, the portion at which the third mark 6C and/or third hole 47 is provided is one example of the third hole portion, the downstream end part of the first channel 29 (position of first hole 43) is one example of the first portion, the upstream end part of the first channel 29 (position of third hole 47) is one example of the second portion, the mixing chamber 55 is one example of the space, the upstream end of the common channel 49 is one example of the inlet, and the first hole forming member 115A, second hole forming member 115B, and third hole forming member 115C are examples of the first member, second member, and third member.

Further, the step of supplying the liquid specimen La in FIG. 5B is one example of the first supplying step, the step of forming the first hole 43 in FIG. 5C is one example of the first communication step, the step of supplying the liquid Le in FIG. 6B is one example of the second supplying step, the step of forming the second hole 45 in FIG. 6C is one example of the second communication step, the step of closing the first hole 43 in FIG. 6A is one example of the blockade step, the step of forming the third hole 47 in FIG. 6A is one example of the blockade step, and the step of forming the third hole 47 in FIG. 6A is one example of the third communication step. The blood or diluted blood is one example of the liquid specimen. In a case where the diluted blood is the liquid specimen, the blood is one example of the undiluted solution of the liquid specimen, PBS is one example of the liquid diluting the liquid specimen, and the rinse water is one example of the washing solution. In the step of supplying the liquid specimen La in FIG. 5B, the step of supplying PBS is one example of the diluent supplying step, and the step of supplying the rinse water Ld in FIG. 5E is one example of the washing solution supplying step.

The present invention is not limited to the above embodiment or modifications and may be executed in various aspects.

The sensor is not limited to one utilizing SAW. For example, it may be one utilizing surface plasmon resonance or may be one utilizing vibration of a quartz resonator. Further, the sensor is not limited to a biosensor. From another viewpoint, the detection portion is not limited to one in which an aptamer is arranged. For example, the sensor element may be one having an electrode for measuring pH (potential hydrogen) based on the change of the potential as well.

Further, the sensor may be used for any purpose. In other words, the type of the specimen (liquid specimen) may be any type as well. For example, the type of specimen may be a body fluid (for example blood), may be a beverage, may be a drug solution, or may be water which is not pure water (for example, seawater, lake water, or underground water). Further, for example, the type of specimen may be one containing water or may be one containing oil. Further, for example, the type of specimen may be a solution or may be a sol.

The channel in which the liquid specimen flows may be suitably constituted other than by those exemplified in the embodiment and modifications. For example, in the embodiment, the inflow port 3 was opened in the upper surface of the package 11, but may be opened in the end face (side face) of the package 11 as well. This is true also for the first to third holes. Further, for example, in the embodiment, the widths of the inflow portion 31a and the outflow portion 31c were made narrower than the width of the element arrangement portion 31b, but may be equal to the width of the element arrangement portion 31b as well.

The package is not limited to one constituted by stacking a plurality of base substrates (members). For example, the package may be integrally formed as well.

In the embodiment, the first channel was utilized as a channel for retracting an ingredient which is not to be tested and the second channel was utilized as a channel for making the ingredient to be tested flow in. However, the sensor elements may be arranged in both of the first channel and second channel and test for the liquid specimen may be carried out in the two channels. For example, in the embodiment, the sensor element may be arranged not only in the second channel, but also in the first channel, a test may be carried out for the blood plasma flowing into the first channel, and a test may be carried out for the blood cell ingredient flowing into the second channel.

Three or more channels may be provided downstream of the filter as well. In this case, for example, after supplying the liquid specimen to the filter, liquids which react with specific ingredients in the liquid specimen are supplied in order to make three or more ingredients flow into the different channels.

The hole portions (first to third hole portions) in the package indicate the portions for opening holes capable of communicating the inside and outside of the package. It is not always necessary to already form the holes. For example, as in the embodiment, so far as the portions at which the holes are to be formed are indicated by marks, the portions constitute the hole portions. Further, for example, if specific portions in the package become thin and so on in order to easily open the holes, these portions constitute the hole portions.

The use of the sensor (communication of the channels and an external portion according to the formation of holes or release of blockades) may be carried out under atmospheric pressure and an air atmosphere, for example, used in an ordinary room. Note that, in this case, the communication between the channels and the external portion can be grasped as release of the channels which have been sealed into atmospheric air. Further, the sensor may be used under an environment other than atmospheric pressure and an air atmosphere as well. For example, the sensor may be used under a nitrogen atmosphere in order to check the property of the liquid specimen which is easily oxidized before oxidation.

### REFERENCE SIGNS LIST

1 . . . sensor, 7 . . . filter, 9 . . . sensor element, 11 . . . package, 29 . . . first channel, 31 . . . second channel, and La . . . liquid specimen.

1. A liquid specimen sensor, comprising:
a filter configured to filter a liquid specimen;
a first channel which is located downstream relative to the filter and is configured to be a capillary;
a second channel which is located downstream relative to the filter and is configured to be a capillary; and
a sensor element which is located in at least one of the first channel and the second channel and configured to output a signal in accordance with an ingredient of the liquid specimen.

2. The liquid specimen sensor according to claim 1, wherein the first channel comprises a first hole portion capable of communicating a first portion with an external portion.

3. The liquid specimen sensor according to claim 1, wherein
the sensor element is located in the second channel, and
the sensor element comprises a second hole portion capable of communicating a portion located more downstream than the sensor element with an external portion.

4. The liquid specimen sensor according to claim 2, wherein the first channel further comprises a third hole portion capable of communicating a second portion located more upstream than the first portion with an external portion.

5. The liquid specimen sensor according to claim 1, wherein a capillary force of the first channel is larger than a capillary force of the second channel.

6. The liquid specimen sensor according to claim 1, wherein a capillary force of the second channel is larger than a capillary force of the first channel.

7. The liquid specimen sensor according to claim 1, wherein
each of the first channel and the second channel is connected to the filter, and
a portion in the first channel which is connected to the filter and a portion in the second channel which is connected to the filter are separated from each other.

8. The liquid specimen sensor according to claim 1, further comprising a common channel which is connected to the filter, wherein the first channel and the second channel are connected to a downstream end part of the common channel.

9. The liquid specimen sensor according to claim 1, wherein the first channel comprises a porous material thereinside.

10. The liquid specimen sensor according to claim 1, further comprising a common channel which is connected to the filter and comprises a space in a downstream end part thereof, wherein
each of the first channel and the second channel is connected to the space of the common channel, and
a portion in the first channel which is connected to the common channel and a portion in the second channel which is connected to the common channel are separated from each other.

11. The liquid specimen sensor according to claim 10, wherein the common channel comprises a cross-sectional area in the space larger than a cross-sectional area of an inlet through which the liquid specimen flows into the common channel.

12. The liquid specimen sensor according to claim 1, wherein
the second channel is connected to the filter, and
in a portion in which the second channel and the filter are connected, a thickness of the second channel is smaller than a thickness of the filter.

13. The liquid specimen sensor according to claim 1, wherein
the second channel is connected to the filter, and
in a perspective plan view, a width of a connecting part with the filter in the second channel is larger than a width of a portion located more downstream than the connecting part.

14. The liquid specimen sensor according to claim 1, wherein at least a portion of the first channel and at least a portion of the second channel are different from each other in the positions in a thickness direction.

15. A liquid specimen sensor unit, comprising:
a liquid specimen sensor according to claim 1; and
a reader to which the liquid specimen sensor can be attached and from which the liquid specimen sensor can be detached.

16. The liquid specimen sensor unit according to claim 15, wherein the reader comprises a first member capable of communicating a first portion in the flow direction in the first channel with an external portion.

17. The liquid specimen sensor unit according to claim 15, wherein
the sensor element is located in the second channel, and
the reader further comprises a second member capable of communicating a portion located more downstream than the sensor element in the second channel with an external portion.

18. The liquid specimen sensor unit according to claim 16, wherein the reader further comprises a third member capable of communicating a second portion located more upstream than the first portion in the first channel with an external portion.

19. A liquid specimen testing method, comprising:
a first supplying step of supplying a liquid specimen to a filter in a liquid specimen sensor, the liquid specimen sensor comprising
the filter filtering the liquid specimen,
a first channel and a second channel which are located downstream relative to the filter and are configured to be capillaries, and
a sensor element which is located in the second channel and outputs a signal in accordance with an ingredient of the liquid specimen;
a first communication step of communicating a first portion in the flow direction in the first channel with an external portion;
a second supplying step of supplying a liquid capable of reacting with the liquid specimen to the filter after the first supplying step and the first communication step; and
a second communication step of communicating a portion in the second channel which is located more downstream than the sensor element and an external portion after the first supplying step and the first communication step.

20. The liquid specimen testing method according to claim 19, further comprising a closing step of closing a first hole portion communicating the first portion in the first channel with the external portion after the first supplying step and the first communication step and before the second supplying step,

21. The liquid specimen testing method according to claim 19, further comprising a third communication step of communicating a second portion located more upstream than the first portion in the first channel with an external portion after the first supplying step and the first communication step and before the second supplying step.

22. The liquid specimen testing method according to claim 19, further comprising a diluent supplying step of supplying a liquid capable of diluting the liquid specimen to the filter after the first supplying step and before the first communication step.

23. The liquid specimen testing method according to claim 19, further comprising a washing solution supplying step of supplying a washing solution to the filter after the first supplying step and the first communication step and before the second supplying step and the second communication step.
